(19) United States
(12) Patent Application Publication
(10) Pub. No.: US 2015/0342915 A1
(43) Pub. Date: Dec. 3, 2015

(54) INSTANTIZED PRODUCT AND METHODS

(71) Applicants: Suvash KAFLEY, (US); Sara Christine WOSJE, Champlin, MN (US); MILK SPECIALTIES COMPANY, Eden Prairie, MN (US)

(72) Inventors: Suvash Kafley, Eden Prairie, MN (US); Sara Christine Wosje, Champlin, MN (US)

(21) Appl. No.: 14/758,004

(22) PCT Filed: Dec. 23, 2013
(86) PCT No.: PCT/US13/77483

§ 371 Date: Jun. 26, 2015

Related U.S. Application Data
(60) Provisional application No. 61/746,846, filed on Dec. 28, 2012.

(51) Int. Cl. A61K 31/198; A61K 9/107

(52) U.S. Cl. CPC A61K 31/198; A61K 9/107

(57) ABSTRACT

This disclosure describes, in one aspect, a composition that generally includes a hydrophobic biologically active material and an emulsifier coating at least a portion of the hydrophobic biologically active material. In some embodiments, the hydrophobic biologically active material can include an amino acid or a combination of amino acids. In certain embodiments, the amino acids can be leucine, isoleucine, valine, or any combination of two or more of those amino acids. In another aspect, this disclosure describes a composition comprising a liquid and an emulsifier-coated hydrophobic biologically active material. In some embodiments, the liquid and the emulsifier-coated hydrophobic biologically active material are mixed.

### CROSS-REFERENCE TO RELATED APPLICATION

This application claims priority to U.S. Provisional Patent Application Ser. No. 61/746,846, filed Dec. 28, 2012, which is incorporated herein by reference.

### SUMMARY

This disclosure describes, in one aspect, a composition that generally includes a hydrophobic biologically active material and an emulsifier coating at least a portion of the hydrophobic biologically active material.

In some embodiments, the hydrophobic biologically active material can include an amino acid or a combination of amino acids. In certain embodiments, the amino acids can be leucine, isoleucine, valine, or any combination of two or more of those amino acids.

In some embodiments, the emulsifier-coated hydrophobic biologically active material, when mixed with water, produces a substantially cloud-free dispersion. In some embodiments, the emulsifier can include Polysorbate 20, Polysorbate 60, Polysorbate 80, decolored lecithin, a monoglyceride, or a diglyceride.

In another aspect, this disclosure describes a composition comprising a liquid and an emulsifier-coated hydrophobic biologically active material. In some embodiments, after the liquid and the emulsifier-coated hydrophobic biologically active material are mixed, the resulting composition transmits at more than 50% of light at 520 nm. In some embodiments, after the liquid and the emulsifier-coated hydrophobic biologically active material are mixed, at least 67% of the emulsifier-coated hydrophobic biologically active material is sufficiently dissolved or dispersed to pass through a 20 μm filter.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. An overhead photograph of two instantized amino acid compositions 30 seconds after mixing.

FIG. 2. An overhead photograph of four amino acid compositions 5:45 (minutes:seconds) after having been mixed with water. From left to right: a 2:1:1 ratio of leucine:isoleucine:valine (MSG BCAA); a commercially available preparation from Milk Specialties Global, Inc., Eden Prairie, Minn. (MSG iBCAA); a preparation prepared as described in Run 1 of Example 1, using Polysorbate 20 as the wetting agent (MSG Clear iBCAA); and a preparation commercially available as AJIPURE (Ajipure Instant BCAA; Ajinomoto North America, Inc., Raleigh, N.C.).

FIG. 3. A straight-on view photograph of the amino acid compositions shown in FIG. 2 at 6:03 after mixing with water.

FIG. 4. An overhead photograph of the amino acid compositions shown in FIG. 2 at 12:39 after mixing with water.

FIG. 5. A straight-on view photograph of the amino acid compositions shown in FIG. 2 at 18:35 after mixing with water.

FIG. 6. A straight-on view photograph of the amino acid compositions shown in FIG. 2 at 29:23 after mixing with water.

### DETAILED DESCRIPTION OF ILLUSTRATIVE EMBODIMENTS

Instantized materials are products in particulate or powder form that are typically added to a liquid (often a water-based liquid) before the liquid is ultimately used. One common example of an instantized material is a drink mix in which an instantized powder product is added to a liquid to enhance, for example, the nutritional value and/or flavor of the drink.

To increase the palatability of an instantized material to a consumer, the instantized material typically combines with the liquid to which it is added in manner that minimizes the amount of material left uncombined with the liquid, e.g., material that is either left floating on the surface of the liquid or as residual material that collects, uncombined with the liquid, at the bottom of the container holding the liquid. An instantized material may be dissolved, dispersed, or otherwise combined with the liquid the instantized product in the liquid in any suitable manner and can include, for example, agitation and/or stirring.

Exemplary materials that may be instantized include amino acids. Amino acid are often added to liquids to enhance the nutritional value of the liquid food products. Exemplary food products having amino acids added include, but are not limited to, sport drinks, milk products, baby formula, milk replacement products, and the like, whether provided as a liquid ready-to-drink product or as an instant powder. To result in a satisfactory end product, the amino acids should disperse quickly and either dissolve, disperse, or otherwise combine with a liquid. Many amino acids are provided in the physical form of flakes, crystals, or powders. These particles can tend to float on the surface of the liquid rather than dissolve or disperse in the liquid.

More generally, it can be difficult to dissolve and/or disperse hydrophobic materials in aqueous liquids. This disclosure describes, generally, compositions that include a hydrophobic biologically active material that is at least partially coated with an emulsifier. Such compositions, when mixed with an aqueous liquid, readily dissolve, disperse, or otherwise combine with the aqueous liquid to produce a substantially cloud-free product.

Thus, in one aspect, this disclosure describes a composition that includes a hydrophobic biologically active material and an emulsifier coating at least a portion of the hydrophobic biologically active material. Typically, the hydrophobic biologically active material can be provided in any suitable particulate form including, for example, a flake, a pellet, a microparticle, or a nanoparticle. As used herein, the term “particulate” refers to a solid without regard to size or shape. As used herein, a “flake” generally refers to a substantially flat solid form that is much greater in each of two dimensions than in the third. As used herein, the term “microparticle” refers to a particle having an average diameter of about 0.1 μm to 100 μm, while “nanoparticle” refers to a particle having an average diameter of about 1 nm to 100 nm.

In some embodiments, the hydrophobic biologically active material may be ground or milled to reduce the size of the particles. The final size of the particles may be any suitable size. Generally speaking, reducing the size of the particles increases the total surface area of the hydrophobic biologically active material for subsequent coating with the emulsifier.

In some embodiments, the hydrophobic biologically active material may be ground or milled to produce particles that have a maximum average diameter of no more than 1000 μm such as, for example, a maximum average diameter of no more than 900 μm, no more than 800 μm, no more than 700 μm, no more than 600 μm, no more than 500 μm, no more than 450 μm, no more than 400 μm, no more than 350 μm, no more than 300 μm, no more than 250 μm, no more than 200 μm, no more than 177 μm, no more than 150 μm, no more than 100 μm, no more than 75 μm, no more than 50 μm, no more than 25 μm, no more than 10 μm, no more than 5 μm, no more than 1 μm, no more than 0.5 μm, or no more than 0.01 μm. In some embodiments, the hydrophobic biologically active material may be ground or milled to produce particles that have a minimum average diameter of at least 1 nm such as, for example, at least 10 nm, at least 100 nm, at least 1 μm, at least 10 μm, at least 25 μm, at least 50 μm, at least 100 μm, at least 200 μm, at least 250 μm, or at least 500 μm. The size of the particles also can be characterized by any range that includes, as endpoints, any combination of a minimum average diameter identified above and any maximum average diameter identified above that is greater than the minimum average diameter. For example, in some embodiments, the particles can have an average diameter of from about 100 μm to about 250 μm.

In some embodiments, the hydrophobic biologically active material may be ground or milled to produce particles having a maximum size no larger than 40 mesh such as, for example, no larger than 50 mesh, no larger than 60 mesh, no larger than 70 mesh, or no larger than 80 mesh. In some embodiments, the hydrophobic biologically active material may be ground or milled to produce particles having a minimum size no smaller than 100 mesh, no smaller than 80 mesh, no smaller than 70 mesh, no smaller than 60 mesh, or no smaller than 50 mesh. The size of the particles also can be characterized by any range that includes, as endpoints, any combination of a minimum size identified above and any maximum size identified above that is greater than the minimum size.

In some embodiments, the hydrophobic biologically active material can include an amino acid or a combination of amino acids such as, for example, blended mixture of amino acids. Exemplary amino acids that can be included as a hydrophobic biologically active material include, for example, leucine, isoleucine, valine, methionine, tryptophan, cysteine, alanine, tyrosine, phenylalanine, histidine, threonine, serine, or proline. In certain embodiments, hydrophobic biologically active material includes leucine, isoleucine, valine, a combination of leucine and isoleucine, a combination of leucine and valine, a combination of isoleucine and valine, or a combination of leucine, isoleucine, and valine.

The composition generally includes an emulsifier that coats at least a portion of the hydrophobic biologically active material to form an emulsifier-coated hydrophobic biologically active material, also referred to herein as an “instantized product.” The emulsifier is generally selected to form a substantially cloud-free dispersion in water. In some of these embodiments, the instantized product likewise produces a substantially cloud-free dispersion when mixed with water. The emulsifier generally reduces liquid surface tension, allowing the coated hydrophobic biological material to more readily enter the liquid, thereby allowing more rapid and/or more complete dissolution and/or dispersion in the liquid.

In some embodiments, the instantized product, when combined with distilled water at room temperature and stirred for 20 seconds, will result in a liquid composition that is capable of transmitting more than 50% of light at 520 nm such as, for example, at least 52%, at least 54%, at least 56%, at least 58%, at least 60%, at least 62%, at least 64%, at least 66%, at least 68%, at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, or at least 99% of light at 520 nm.

Suitable emulsifiers include, for example, Polysorbate 20, Polysorbate 60, Polysorbate 80, decolored lecithin, a monoglyceride, or a diglyceride.

Generally, the emulsifier may be applied to the surface of the hydrophobic biologically active material in any suitable manner. Exemplary methods include, for example, spraying the emulsifier onto dry particles of the hydrophobic biologically active material, then agitating the mixture sufficiently to produce a thorough intermixture.

The emulsifier may be added in amount to be a minimum of at least 0.5% by weight of the final emulsifier-coated instantized product such as, for example, at least 1.0%, at least 1.5%, at least 2.0%, at least 2.5%, at least 3.0%, at least 3.5%, at least 4.0%, at least 4.5%, at least 5.0%, at least 5.5%, at least 6.0%, at least 6.5%, at least 7.0%, at least 7.5%, at least 8.0%, at least 8.5%, at least 9.0%, at least 9.5%, or at least 10.0% of the final instantized product by weight. In some embodiments, the emulsifier may be added in amount to be a maximum of no more than 10.0% by weight of the final instantized product such as, for example, no more than 9.5%, no more than 9.0%, no more than 8.5%, no more than 8.0%, no more than 7.5%, no more than 7.0%, no more than 6.5%, no more than 6.0%, no more than 5.5%, no more than 5.0%, no more than 4.5%, no more than 4.0%, no more than 3.5%, no more than 3.0%, no more than 2.5%, no more than 2.0%, no more than 1.5%, no more than 1.0%, or no more than 0.5% of the final instantized product by weight. The amount of emulsifier in the final instantized product also can be characterized by any range that includes, as endpoints, any combination of a minimum amount identified above and any maximum amount identified above that is greater than the minimum amount. For example, in some embodiments, the emulsifier can be added to the hydrophobic biologically active material to be about 3% to about 5% of the final instantized product by weight.

In some embodiments, the instantized product can further include an antifoam agent. An antifoam agent may be include in circumstances when, for example, strong agitation is used to blend the instantized product with water or other liquid. An antifoam agent may reduce the extent to which foam is produced from the agitation. The antifoam agent also may break down foam that does form. Suitable antifoam agents include, for example, conventional silicon-based food safe antifoam agents, water-based food safe antifoaming agents, oil-based food safe antifoaming agents, or any combination of two or more suitable antifoam agents.

When present, the antifoam agent can be present in a minimum amount at least 0.1% of the weight of the emulsifier such as, for example, at least 0.2%, at least 0.5%, at least 0.75%, at least 1.0%, at least 2.0%, at least 3.0%, at least 4.0%, at least 5.0%, at least 6.0%, at least 7.0%, at least 8.0%, or at least 9.0% of the weight of the emulsifier. The antifoam agent may be present in a maximum amount of no more than 10% of the weight of the emulsifier such as, for example, no more than 9%, no more than 8%, no more than 7%, no more than 6%, no more than 5%, no more than 4%, no more than 3%, or no more than 2% of the weight of the emulsifier. The amount of antifoam agent also can be characterized by any range that includes, as endpoints, any combination of a minimum amount identified above and any maximum amount identified above that is greater than the minimum amount. For example, in some embodiments, the antifoam agent can be present in amount of 1% to 10% of the weight of the emulsifier. In other embodiments, the antifoam agent can be present in a range of, for example, from 1% to 7% of the weight of the emulsifier or, for example, from 1% to 5% of the weight of the emulsifier.

Generally, the the antifoam agent is added can be blended with the emulsifier, applied to the hydrophobic biologically active material prior to the emulsifier, or may be added after the emulsifier has been applied to the hydrophobic biologically active material. The antifoam agent is typically added to the instantized product under any suitable conditions to decrease the production of foam.

In another aspect, this disclosure describes a composition that includes an emulsifier-coated hydrophobic biologically active material mixed with the liquid. In use, the instantized product may be mixed with any suitable aqueous liquid. Suitable aqueous liquids include, for example, water, milk, infant formula, a sport drink, an energy drink, coffee, tea, fruit juice, protein shakes, and/or meal-replacement beverages. The instantized product may be mixed with the liquid in any suitable manner including, for example, being dissolved in or dispersed in the liquid. Mixing the instantized product and the liquid can include, for example, agitation, stirring, blending, and/or heating in order to promote the dissolution and/or dispersion of the instantized product in the liquid.

A common feature of conventional compositions that include, for example, hydrophobic amino acids is that when mixed with a liquid, some of the particles fail to dissolve or disperse in the liquid. In some cases, the undissolved and/or undispersed particles can float on the surface of the liquid. Although amino acids typically have a specific gravity greater than that of common liquids, such as water and milk, to which the amino acids are added, an amino acid can tend to float on the surface of the liquid because of, at least in part, the shape of the amino acid particles and/or the surface tension of the liquid. When the used in a product such as, for example, a drink mix, consumers often find undissolved and/or undispersed particles on the surface of the liquid unappealing. Consequently, a composition that improves the extent to which a hydrophobic biologically active material—e.g., hydrophobic amino acids—dissolves or disperses in a liquid can improve consumer reaction to such a product.

Also, in some embodiments, the instantized product can cross the surface of and enter the liquid in a period of a few seconds such as, for example, a period of about 3 to about 20 second such as, for example, a period of about 3 to about 10 seconds.

Also, in some embodiments, the instantized product described herein, when combined with water at room temperature and stirred for 20 seconds, result in a liquid composition in which the instantized product is sufficiently dissolved or dispersed in the liquid so that at least 67% of the instantized product in the liquid composition, by weight, passes through a 20 μm filter (e.g., grade P8 filter paper) such as, for example, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the instantized product passes through such as filter.

A liquid composition that includes the dissolved and/or dispersed instantized product can include one or more additional ingredients such as, for example, a vitamin, a mineral, a nutritional supplement, a sweetener, a flavorant, a thickening agent, or any combination of two or more such ingredients.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Throughout this disclosure, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

The present invention is illustrated by the following examples. It is to be understood that the particular examples, materials, amounts, and procedures are to be interpreted broadly in accordance with the scope and spirit of the invention as set forth herein.

### EXAMPLES

### Example 1

L-Valine (4830 g), L-Isoleucine (4830 g), and L-Leucine (9660 g) were blended together to uniformity with no agglomeration.

The amino acid powder blend was mixed with a wetting agent, either sorbitan monolaurate, ethoxylated (T-MAZ 20, BASF Corp., Florham Park, N.J.) or lecithin (SOLEC E, Ultimate Source, Inc., Dallas, Tex.) diluted with vegetable oil (ConAgra Foods, Inc., Omaha, Nebr.). The mixing was performed using a fluidizing paddle blender (OPTIMABLEND FPB2, American Process Systems, Eirich Machines Inc., Gurnee, Ill.), set at full speed (60 Hz, 105 rpm) for 60 seconds.

Run 1: Sorbitan monolaurate, ethoxylated (966 g, 5% dry weight of amino acid blend) was added to a three-gallon canister and pressurized to 100 psig. Air pressure at 38 psig was connected to the other port on the atomizing spray nozzle. The wetting agent transferred over 83 seconds to the amino acid powder blend by spraying into the fluidizing paddle blender. The product was post-mixed (60 Hz, 105 rpm) for two minutes, four minutes, or six minutes. Final bulk density of the mixture was 0.26 kg/L.

Run 2: Sorbitan monolaurate, ethoxylated (580 g, 3% dry weight of amino acid blend) was added to a three-gallon canister and pressurized to 100 psig. Air pressure at 30 psig was connected to the other port on the atomizing spray nozzle. The wetting agent transferred over 47 seconds to the amino acid powder blend by spraying into the fluidizing paddle blender. The product was post-mixed (60 Hz, 105 rpm) for two minutes, four minutes, or six minutes. Final bulk density of the mixture was 0.272 kg/L.

Run 3: Lecithin was diluted with vegetable oil (3 parts lecithin to 1 part oil). The lecithin-oil mixture (578 g lecithin, 3% dry weight of amino acid blend, plus 192 g oil) was added to a three-gallon canister and pressurized to 100 psig. Air pressure at 40 psig was connected to the other port on the atomizing spray nozzle. The wetting agent transferred over 230 seconds to the amino acid powder blend by spraying into the fluidizing paddle blender. The product was post-mixed (60 Hz, 105 rpm) for two minutes. Final bulk density of the mixture was 0.3025 kg/L.

Each mixture was mixed with water in a vessel and analyzed for its solubility/dispersability. Results are summarized in Table 1.

TABLE 1

| | Characteristics |
|---|---|
| Run 1 | |
| 2 minutes | Partially sank, part floated. No agglomerates. |
| 4 minutes | Dispersed in water, became clear, few particles; some |
| | foam at surface. |
| 6 minutes | Dissolved readily, some foam at surface |
| Run 2 | |
| 2 minutes | Dissolved slowly in water; eventually went clear, some |
| | floaters; some foam at surface. |
| 4 minutes | Similar to 2 minutes, with a reduction in foam. |
| 6 minutes | Similar to 2 minutes and 4 minutes, less foam than 4 minutes. |
| Run 3 | |
| 2 minutes | Dissolved slowly in water, yellow particles present. |

### Example 2

Samples of four branched chain amino acid (BCAA) compositions were prepared to monitor visual clarity over time. One composition (labeled MSG BCAA) was prepared as a 2:1:1 ratio of leucine:isoleucine:valine. The second composition (labeled MSG iBCAA) is commercially available from Milk Specialties Global, Inc., Eden Prairie, Minn. The third composition (labeled MSG Clear iBCAA) was prepared as described in Run 1 of Example 1, above, using Polysorbate 20 as the wetting agent. The fourth composition (labeled Ajipure Instant BCAA) is commercially available as AJIPURE, Ajinomoto North America, Inc., Raleigh, N.C.

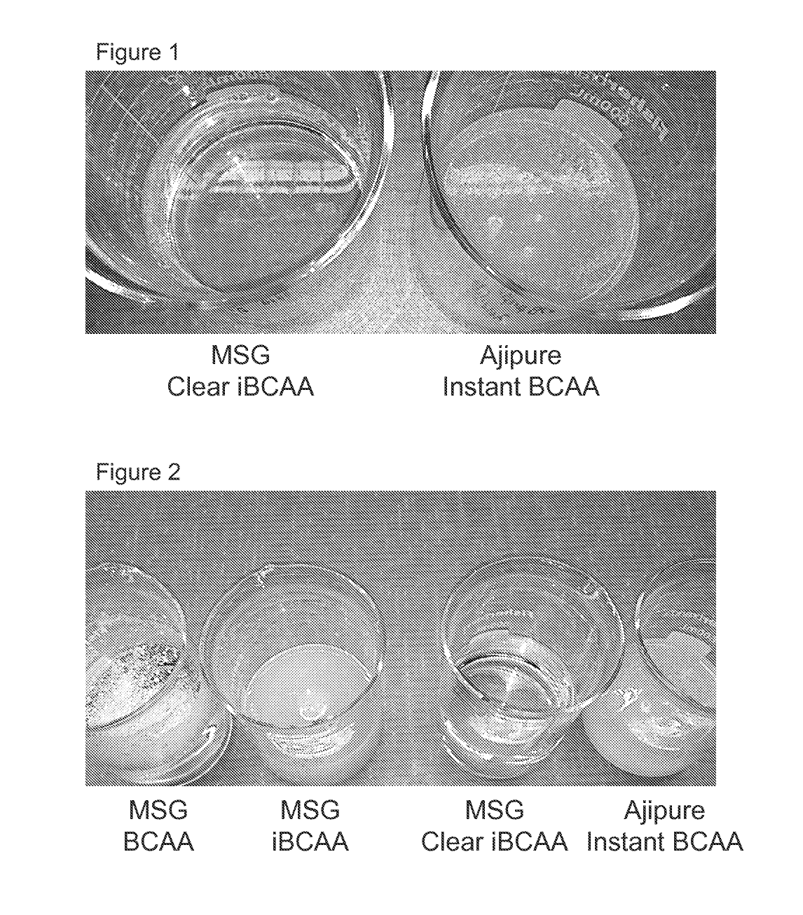

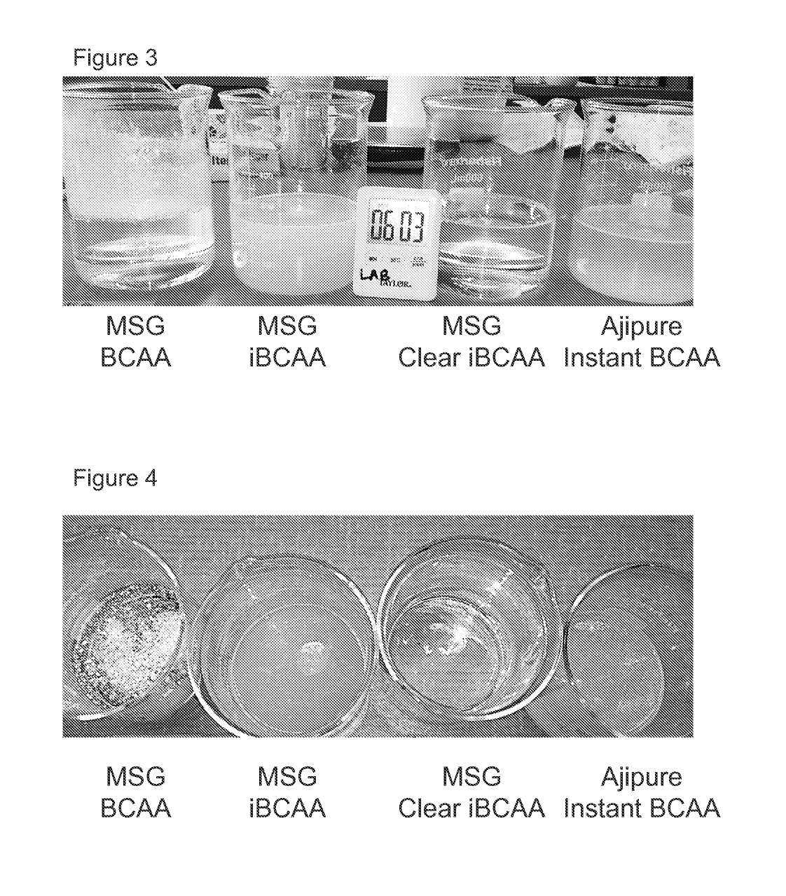

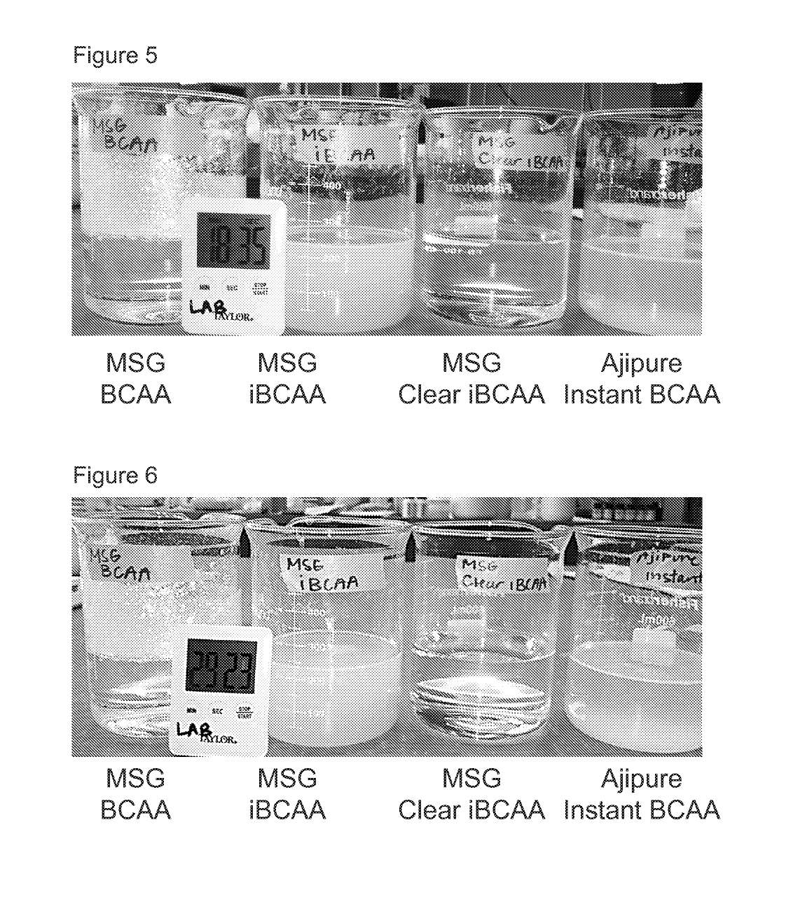

A 2.5% solution of each sample was made by combining 5 g of BCAA in 190 g of distilled water, then stirring with a plastic spoon for 20 seconds at room temperature. The solutions were then allowed to settle and were photographed at various time points after stirring was completed. Results, with time elapsed from the end of the stirring, are shown in FIG. 1-6.

### Example 3

Three branched chain amino acid compositions were tested for the percent light transmittance through a 2.5% solution of BCAA in water: AJIPURE (Ajinomoto North America, Inc., Raleigh, N.C.), Clear iBCAA prepared as described in Example 2 (MSG Clear iBCAA), and Clear iBCAA ground to 80 mesh.

A 2.5% solution of each composition was made by combining 5 g of BCAA in 195 g of distilled water, then stirring with a plastic spoon for 20 seconds at room temperature. The solutions were then allowed to settle for two minutes and the transmittance was measured on a Genesys 10S spectrophotometer (Thermo Fisher Scientific, Inc., Waltham Mass.) at wavelength 520 nm. Results are shown in Table 2.

TABLE 2

| BCAA Composition | Optical transmittance at 520 nm |
|---|---|
| AJIPURE | 50% |
| Clear iBCAA (Regular, 60 mesh) | 99% |
| Clear iBCAA (Ground, 80 mesh) | 99% |

### Example 4

The three branched chain amino acid compositions of Example 3 were tested for the amount of product from a 5% solution of BCAA that dissolved in water. A 5% solution of each composition was made by combining 10 g of BCAA in 190 g of distilled water, then stirring with a plastic spoon for 20 seconds at room temperature. The solutions were then allowed to settle for two minutes, and sent through a Buchner funnel with a filter paper (P8 filter paper, Thermo Fisher Scientific, Inc., Waltham, Mass.) until no visible water was present. The wet filters with retained BCAA were then dried with a SMART Turbo Microwave Moisture/Solids Analyzer (CEM Corp., Matthews, N.C.) to 110° C. until it could not detect any more moisture. The dried filters containing BCAA were then weighed and the difference between the weight of the filter paper and the remaining BCAA was recorded. The percentage of each composition that passed through the filter is shown in Table 3.

TABLE 3

| BCAA Composition | % filter pass through |
|---|---|
| AJIPURE | 66.73% |
| Clear iBCAA (60 mesh) | 83.38% |
| Clear iBCAA (80 mesh) | 88.82% |

### Exemplary Embodiments

Embodiment 1. A composition comprising a hydrophobic biologically active material; and an emulsifier coating at least a portion of the hydrophobic biologically active material.

Embodiment 2. The composition of Embodiment 1 wherein the hydrophobic biologically active material comprises a particle, flake, or pellet.

Embodiment 3. The composition of Embodiment 1 wherein the hydrophobic biologically active material comprises an amino acid.

Embodiment 4. The composition of Embodiment 1 wherein the hydrophobic biologically active material comprises a combination of amino acids.

Embodiment 5. The composition of Embodiment 3 or Embodiment 4 wherein the amino acid comprises leucine, isoleucine, valine, methionine, tryptophan, cysteine, alanine, tyrosine, phenylalanine, histidine, threonine, serine, or proline.

Embodiment 6. The composition of Embodiment 5 wherein the amino acid comprises leucine, isoleucine, and valine.

Embodiment 7. The composition of Embodiment 1 wherein the emulsifier produces a substantially cloud-free dispersion in water.

Embodiment 8. The composition of any preceding Embodiment wherein the emulsifier-coated hydrophobic biologically active material, when mixed with water, produces a substantially cloud-free dispersion.

Embodiment 9. The composition of any preceding Embodiment further comprising a liquid, wherein the emulsifier-coated hydrophobic biologically active material is mixed with the liquid.

Embodiment 10. The composition of Embodiment 9 wherein the emulsifier-coated hydrophobic biologically active material dissolves in or is dispersed in the liquid.

Embodiment 11. The composition of Embodiment 9 or Embodiment 10 wherein the liquid composition transmits at more than 50% of light at 520 nm.

Embodiment 12. The composition of any one of Embodiments 9-11 wherein at least 67% of the emulsifier-coated hydrophobic biologically active material passes through a 20 μm filter.

Embodiment 13. The composition of any one of Embodiments 7-12 wherein the emulsifier comprises Polysorbate 20, Polysorbate 60, Polysorbate 80, decolored lecithin, a monoglyceride, or a diglyceride.

Embodiment 14. The composition of any preceding Embodiment further comprising an antifoam agent.

Embodiment 15. The composition of Embodiment 14 wherein the antifoam agent is present in an amount from 1% to 10% of the weight of the emulsifier.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
a hydrophobic biologically active material; and
an emulsifier coating at least a portion of the hydrophobic biologically active material.

2. The composition of claim 1 wherein the hydrophobic biologically active material comprises a particle, flake, or pellet.

3. The composition of claim 1 wherein the hydrophobic biologically active material comprises an amino acid.

4. The composition of claim 1 wherein the hydrophobic biologically active material comprises a combination of amino acids.

5. The composition of claim 3 wherein the amino acid comprises leucine, isoleucine, valine, methionine, tryptophan, cysteine, alanine, tyrosine, phenylalanine, histidine, threonine, serine, or proline.

6. The composition of claim 5 wherein the amino acid comprises leucine, isoleucine, and valine.

7. The composition of claim 1 wherein the emulsifier produces a substantial cloud-free dispersion in water.

8. The composition of claim 1 wherein the emulsifier-coated hydrophobic biologically active material, when mixed with water, produces a substantially cloud-free dispersion.

9. The composition of claim 1 further comprising a liquid, wherein the emulsifier-coated hydrophobic biologically active material is mixed with the liquid.

10. The composition of claim 9 wherein the emulsifier-coated hydrophobic biologically active material dissolves in or is dispersed in the liquid.

11. The composition of claim 9 wherein the liquid composition transmits at more than 50% of light at 520 nm.

12. The composition of claim 9 wherein at least 67% of the emulsifier-coated hydrophobic biologically active material passes through a 20 μm filter.

13. The composition of claim 7 wherein the emulsifier comprises Polysorbate 20, Polysorbate 60, Polysorbate 80, decolored lecithin, a monoglyceride, or a diglyceride.

14. The composition of claim 4 wherein the amino acid comprises leucine, isoleucine, valine, methionine, tryptophan, cysteine, alanine, tyrosine, phenylalanine, histidine, threonine, serine, or proline.

15. The composition of claim 4 wherein the amino acid comprises leucine, isoleucine, and valine.

16. The composition of claim 10 wherein the liquid composition transmits at more than 50% of light at 520 nm.

17. The composition of claim 10 wherein at least 67% of the emulsifier-coated hydrophobic biologically active material passes through a 20 μm filter.

18. The composition of claim 11 wherein at least 67% of the emulsifier-coated hydrophobic biologically active material passes through a 20 μm filter.
